(19) United States
(12) Patent Application Publication
(10) Pub. No.: US 2007/0258903 A1
(43) Pub. Date: Nov. 8, 2007

(54) Methods, compositions and devices for treating lesioned sites using bioabsorbable carriers

(76) Inventors: Lothar W. Kleiner, Los Altos, CA (US); Syed Hossainy, Fremont, CA (US); Irina Astafieva, Palo Alto, CA (US); Stephen Pacetti, San Jose, CA (US); Thierry Glauser, Redwood City, CA (US); Jessica DesNoyer, San Jose, CA (US); Florian N. Ludwig, Mountain View, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/416,860

(22) Filed: May 2, 2006

(51) Int. Cl. A61K 39/00; A61K 49/10

(52) U.S. Cl. 424/9.3; 424/426; 424/133.1; 424/9.6; 514/2; 977/906

(57) ABSTRACT

Methods and compositions for the sustained release of treatment agents to treat an occluded blood vessel and affected tissue and/or organs are disclosed. Porous or non-porous bioabsorbable glass, metal or ceramic bead, rod or fiber particles can be loaded with a treatment agent, and optionally an image-enhancing agent, and coated with a sustained-release coating for delivery to an occluded blood vessel and affected tissue and/or organs by a delivery device.

### FIELD OF INVENTION

Percutaneous treatments, methods and compositions for body vessels, tissues and organs.

### BACKGROUND OF INVENTION

“Arteriosclerosis” refers to the thickening and hardening of arteries. “Atherosclerosis” is a type of arteriosclerosis in which fatty substances, cholesterol, cellular waste product, calcium and fibrin build up in the inner lining of a physiological vessel. The resultant build-up, or occlusion, is commonly referred to as plaque. It is generally believed that atherosclerosis begins with damage to the inner arterial wall resulting in a lesion. The damaged site attracts substances such as fats, platelets, cholesterol, cellular waste products and calcium which are deposited on the damaged site. In turn, these substances stimulate the cells of the inner arterial wall to produce other substances which accumulate and cause more damage. These accumulations also inhibit the rate of blood flow which can damage tissue and/or organs adjacent to or downstream from the damaged vessel.

Mechanical methods can be used to treat plaque build-up in occluded blood vessels. Angioplasty and stent deployment are examples of such mechanical methods. In one stent deployment method, an absorbable metal stent can be used to treat stenosis. “Stenosis” refers to a narrowing or constriction of the diameter of a vessel. See, e.g., Eggebrecht, H. et al., Novel Magnetic Resonance-Compatible Coronary Stent, Circulation. 2005; 112:e303-e304; Heublein, B. et al., Biocorrosion of magnesium alloys: a new principle in cardiovascular implant technology?, Heart. 2003; 89:651-656.

Blood flow is the flow of blood through the cardiovascular system and can be defined by the formula F equals ΔP/R wherein R is (vL/r⁴)(8/Π) wherein F is blood flow, P is pressure, R is resistance, v is fluid viscosity, L is length of tube and r is radius of tube. Blood leaving the heart is typically at its highest pressure, or about 100 mmHg for a healthy individual, and blood returning to the heart is typically at its lowest pressure, or about 5 mmHg for a healthy individual. Blood flow undergoes both turbulent and laminar flow, and subjects the blood vessel walls to pressure. “Laminar flow” is smooth fluid motion. “Turbulent flow” is disrupted fluid motion. Laminar flow generally takes place adjacent to the walls of a blood vessel, while turbulent flow generally occurs at higher flow velocities, and takes place towards the middle of a blood vessel.

Therapies involving the use of delivery devices to deliver treatment agents are known to have a beneficial effect on vulnerable plaque, other harmful build-up in the inner wall of a diseased blood vessel and/or damaged tissue and/or organs fed by a diseased blood vessel. Thus, in theory, blood vessel occlusions and resultant damaged tissue and/or organs can be treated by releasing a treatment agent on or near the treatment site using a mechanical instrument such as a catheter. Because of the blood flow and the pressure exerted by the flow of blood on the walls of the blood vessel, however, all or substantially all of the treatment agent can be washed away from the treatment site resulting in minimal, if any, beneficial effect at the treatment site.

Local therapy involving the use of an implantable medical device is also known to have a beneficial effect on vulnerable plaque and other harmful build-up in the inner wall of a diseased blood vessel. Recently, the use of agents incorporated within an implantable medical device, such as a stent, has been used to treat the side effects of stent implantation, such as restenosis and inflammation. “Restenosis” is the reoccurrence of stenosis in a blood vessel or heart valve after it has been treated with apparent success. Such a system, typically called a drug-eluting stent or “DES stent”, can generally include a hydrophobic polymer carrier and an agent dispersed throughout a coating solution and then applied to the stent for sustained release thereof. For hydrophilic agents, the initial burst rate can be greater than 40 percent (%) wherein the hydrophilic agent is released within a period of less than 24 hours. Generally, the DES stent can release the agent throughout a period of at least 30 days. “Burst” refers to the amount of drug released in one day or any short duration divided by the total amount of drug (which is released for a much longer duration). For example, in the Xience™ V Drug Eluting Coronary Stent, a product developed by Abbott Vasculature, Santa Clara, Calif., the burst is about 25% to 30% (amount released in 1 day), with the remaining drug released over a thirty day period. For hydrophilic drugs, the burst can usually much higher. Thus, challenges to such systems include reducing the burst in DES systems when hydrophilic agents are incorporated therein.

### SUMMARY OF INVENTION

Methods, compositions and devices for the sustained release of treatment agent to treat an occluded blood vessel and affected tissue and/or organs are disclosed herein.

According to some embodiments, a method includes percutaneously introducing a delivery device into a blood vessel from a point outside a patient and delivering at least one substance to a treatment site within a lumen of a blood vessel by a sustained-release carrier. The carrier can be a bioabsorbable glass, a bioabsorbable metal or a bioabsorbable ceramic. The carrier can be porous or non-porous. The substance can be at least one of a cellular component, a treatment agent or an image-enhancing agent. In addition, the carrier can be coated with a sustained-release coating substance. The substance can be present in at least one pore of the carrier. The carrier can be a first carrier that, at the time of delivery, comprises part of a second carrier.

According to some embodiments, a method of manufacturing a composition includes: loading a substance into a carrier device; after the loading, coating the carrier device with a coating substance; and after the coating, suspending the carrier device in a solution.

According to some embodiments, a composition includes one of a bioabsorbable metal, glass and ceramic carrier; and a treatment agent loaded within or on the bioabsorbable carrier.

According to some embodiments, a coating composition for an implantable medical device includes a sustained-release coating including at least one porous carrier that is a bioabsorbable glass, a bioabsorbable metal and a bioabsorbable ceramic, wherein a treatment agent is dispersed within at least one pore of the porous carrier.

### BRIEF DESCRIPTION OF DRAWINGS

FIG. 1 illustrates a diseased blood vessel.

FIG. 2A illustrates an embodiment of a porous biodegradable carrier of the present invention.

FIG. 2B illustrates an alternative embodiment of a porous biodegradable carrier of the present invention.

FIG. 2C illustrates an embodiment of a non-porous biodegradable carrier of the present invention.

FIG. 3 presents a block diagram for preparing the carriers of FIGS. 2A-2C for sustained-release of treatment agent in the body according to the present invention.

FIG. 4 illustrates the blood vessel of FIG. 1 and a first embodiment of a catheter assembly to deliver a treatment agent-loaded carrier to a blood vessel.

FIG. 5 illustrates the blood vessel of FIG. 1 and a second embodiment of a catheter assembly to deliver a treatment agent-loaded carrier to a blood vessel.

FIG. 6 illustrates the blood vessel of FIG. 1 and a third embodiment of a catheter assembly to deliver a treatment agent-loaded carrier to a blood vessel.

FIG. 7A-7C illustrates the blood vessel of FIG. 1 and a fourth embodiment of a catheter assembly to deliver a treatment agent-loaded carrier to a blood vessel.

FIG. 8 illustrates the blood vessel of FIG. 1 and an alternative embodiment for delivering a treatment agent-loaded carrier to a blood vessel using a stent.

FIG. 9 illustrates a schematic illustration of a back view of kidneys and renal blood vessels of body.

### DETAILED DESCRIPTION

The present invention relates to porous or non-porous bioabsorbable metal, glass, ceramic or a combination thereof, particles for use as a carrier for sustained release of a treatment agent(s) to an occluded blood vessel or site-specific areas of tissue and/or organs affected by the occlusion. “Bioabsorbable” is the reabsorption, degradation and breakdown of foreign matter in the body over time. The particles can be spheres, rods, fibers or any other suitable configuration. Moreover, the particles can be formulated such that they dissolve within the body with minimal or no damage to blood vessel walls.

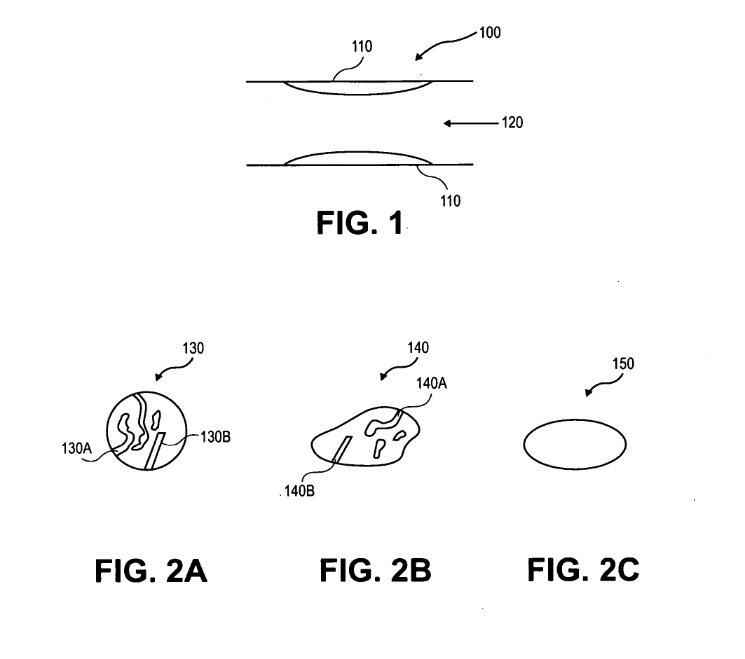

FIG. 1 illustrates an occluded blood vessel 100 with plaque build-up 110 hereinafter interchangeably referred to as a treatment site or injury site. The stenosis or occlusion can result in decreased blood flow through lumen 120. Decreased blood flow delivers fewer nutrients (e.g., oxygenated blood) to tissues fed by the blood vessel resulting in tissue damage or death.

Various methods are employed to reduce the plaque build-up 110 and restore blood flow to affected tissue and/or organs adjacent to or downstream from the damaged vessel 100. In some applications, mechanical methods such as balloon angioplasty or stent delivery can be employed to treat the occlusion. In some applications, treatment agents which directly or indirectly reduce plaque can be employed to treat the occlusion. In some applications, a combination of mechanical methods with treatment agents can be used.

Treatment Agents

In some embodiments, a treatment agent, such as a bioactive agent, can be used to treat an injury site at an occluded blood vessel and to affected tissue and/or organs. Examples of bioactive agents include, but are not limited to, cellular components such as peptides, proteins, oligonucleotides, and the like. For example, the bioactive agent can be apolipoprotein A-1 (Apo A1). APO A1, a constituent of the cholesterol carrier high density lipoprotein (HDL), is involved in reverse cholesterol transport. Its presence can stimulate the release of cholesterol from the walls of an occluded blood vessel.

In some embodiments, a treatment agent can be used to treat an injury site at an occluded blood vessel and to affected tissue and/or organs. In addition to bioactive agents, the treatment agents can include an anti-proliferative, anti-inflammatory or immune modulating, anti-migratory, anti-thrombotic or other pre-healing agent or a combination thereof, and the like. The anti-proliferative agent can be a natural proteineous agent such as cytotoxin or a synthetic molecule or other substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin Cl); all statins (also known as HMG-CoA reductase inhibitors), such as atorvastatin, a combination of atorvastatin and amlodipine, cerivastatin, fluvastatin, lovastatin, mevastatin, pravastatin, rosuvastatin, simvastatin and a combination of simvastatin and ezetimibe; all taxoids such as taxols, docetaxel, and paclitaxel, paclitaxel derivatives; all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbott Laboratories, Abbott Park, Ill.), prodrugs thereof, co-drugs thereof, and combinations thereof.

The anti-inflammatory agent can be a steroidal anti-inflammatory agent, a nonsteroidal anti-inflammatory agent, or a combination thereof, and the like. In some embodiments, anti-inflammatory drugs include, but are not limited to, alclofenac, alclometasone diproprionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, ciclopfrofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen fenclofenac, fenclorac, fendosal, fenpipalone, fentiazac, flazalone, fluazocort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone sodium glycerate, perfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicyclic acid), salicyclic acid, corticosteroids, glucocorticoids, tacrolimus, pimecrolimus, prodrugs thereof, co-drugs thereof, and combinations thereof.

These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable treatment and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g., Adriamycin® from Pharmacia & Upjohn, Peapack, N.J.), and mitomycin (e.g., Mutamycin® from Bristol Myers Squibb Co, Stamford, Conn.). Examples of such antiplatelets, anticoagulants, antifebrin, antithrombins include sodium heparin, low molecular weight heparins, heparinoids, hirudin, argatroban, forskolin, vapiprost, prostacyclin, and prostacyclin analogues, dextran, D-phe-pro-arg-chloromethylketone (synthetic antithrombin), dipyridamole, glycoprotein llb/llla platelet membrane receptor antagonist antibody, recombinant hirudin, thrombin inhibitors such as Angio max å (Biogen, Inc. Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g., Capoten® and Capozide® from Bristol Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other treatment substances or agents which may be appropriate include alpha-interferon, and genetically engineered epithelial cells. The foregoing substances are listed by way of example and are not meant to be limiting. Other treatment agents which are currently available or that may be developed in the future are equally applicable. “Treatment agent” is hereinafter used to refer to bioactive agents and any of the aforementioned agents.

Carriers

In one embodiment, the invention relates to porous or non-porous bioabsorbable metal, glass, ceramic or a combination thereof particles for use as a carrier for sustained release of a treatment agent(s) to an occluded blood vessel or site-specific areas of tissue and/or organs affected by the occlusion. The particles can be spheres, rods, fibers or any other suitable configuration. Moreover, the particles can be formulated such that they dissolve within the body with minimal damage to blood vessel walls. A porous particle can be characterized by its porosity and tortuosity. “Porosity” refers to the ratio of volume of all the pores in a material to the volume of the whole material. “Tortuosity” refers to the winding or twisting of the pores within the particle. Porosity and tortuously are at least two factors which determine the sustained release of the treatment agent(s) once it has reached a treatment site. Other factors which affect sustained release include, but are not limited to, pore size (e.g., microporous or nonporous), thickness of porous membrane, and number and size of pores on surface For example, a porous particle may have a porous sublayer, but very few pores on the surface. A high degree of porosity and tortuosity in a particle mean that the internal surface area within the particle is large resulting in its increased capacity to retain substances. Thus, a greater amount of substance in the pores may allow for a greater timeframe in which the substance may be released.

Porosity can be in a theoretical range of between about 0% to about 100%. In some embodiments, porosity can be in a range of about 0.01% to about 99%. Other factors which can characterize porosity include specific surface area, pore volume, pore size distributions and density. The measured density should be compared to the theoretical density of the material if no pores were present. In some embodiments, porosity can be determined by the formula:
Porosity(%)=V_{bulk}−V/V_{bulk}×100
wherein V_{bulk} is the volume occupied by a selected weight of a substance and V is the true volume of granules, i.e., the space occupied by the substance exclusive of spaces greater than the molecular space. Tortuosity is typically controlled by the manufacturing process of the particles.

Other physical parameters which can be manipulated to control the sustained release of a treatment agent include, but are not limited to, the degree of surface roughness within the pores, the chemical composition of the surface, the pore size gradient and the size distribution of the particles. These factors can influence the amount of treatment agent retained in addition to the degree of retentiveness of a treatment agent within or on the particle. As a result, a greater amount of substance in the pores may allow for a greater timeframe in which the substance may be released.

Chemical parameters which can be manipulated to control the sustained release of a treatment agent include, but are not limited to: the adsorption/chemisorption potential of treatment agents on or within (i.e., within the pores) the particle(s); additional substances loaded on or within the particle(s) which increase or decrease treatment agent retentivity; wettability of the particle(s); interfacial compatibility with the treatment agent and the solvent; and, packaging or encapsulating the treatment agent within other materials with different or similar sustained release characteristics. For example, in some embodiments, substances such as fullerene, activated carbon or metals such as chromium, gold, silver or manganese may be loaded on or within the particle(s). These substances may influence retentivity and interaction characteristics of the treatment agent on or within the particle(s). Also, in some embodiments, the treatment agent may be “packaged” in a biodegradable and/or biostable polymer matrix such as polylactide, poly(ester amide), polyethylene glycol and poly(vinyldiene fluoride-co-hexafluoropropylene) or polybutylmethacrylate (PMBA) before loaded on or within the particle(s). Such packaging can influence the rate of release of the treatment agent depending on the nature of the polymer used. For example, sustained release can be controlled by using polyglycolides (PGA) or polylactide (PLA). PGA is a fast-degrading polymer and has a degradation rate of about 6 months to about 12 months. PLA is a slow-degrading polymer and has a degradation rate of about 12 and about 18 months. It should be appreciated that more than one kind of polymer may be used to tailor degradation rates.

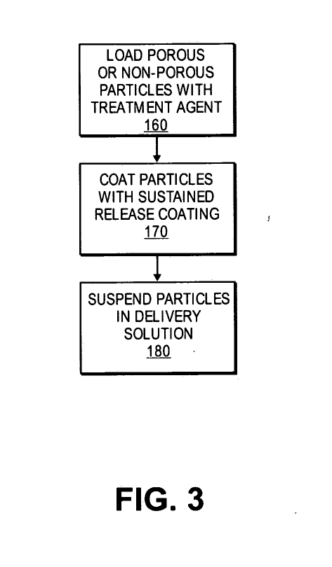

In some embodiments, porous particle 130 of glass, metal or ceramic may be a carrier representatively shown in FIG. 2A. A glass particle may be made of a biocompatible material such as soda lime, silica, borosilicate or aluminosilicate while a metal particle may be made of a magnesium alloy, zinc alloy, or other similarly biocompatible metal. In one embodiment, a glass particle can include iron, magnesium, a soluble ceramic, such as β-tricalcium phosphate (TCP), or any other suitable material which renders it soluble in water over time. In another embodiment, a metal particle can include small amounts of aluminum, manganese, zinc, lithium or other rare earth metals. The particle can include both tortuous pores 130A and non-tortuous pores 130B. The particle can typically be in the range of about 40 μm to 10 μm, preferably 100 nm to 2 μm.

In some embodiments, the pores of the particle can be loaded with at least one treatment agent. For example, the particles can be immersed in a solution of treatment agent for a period of time to allow the treatment agent to fill the pores. In addition, the particles may optionally be loaded with an image-enhancing agent for tracking of the particles by fluoroscopy or magnetic resonance imaging (MRI). For example, in one embodiment, a first number of particles may be loaded with a treatment agent while a second number of particles may be loaded with an image-enhancing agent. The first number and second number of particles may then be combined and delivered in combination. In another embodiment, particles may be combined with both a treatment agent and an image-enhancing agent in the same particles. The image-enhancing agent can include a radiopaque or MRI agent. “Radiopaque” refers to the ability of a substance to absorb x-rays. An MRI agent has a magnetic susceptibility that allows it to be visible with MRI. Representative radiopaque agents may include, but are not limited to, biodegradable metallic particles and particles of biodegradable metallic compounds such as biodegradable metallic oxides, biocompatible metallic salts, iodinated agents and fluorinated dyes. Iodinated radiopaque agents may include, but are not limited to, acetriozate, diatriozate, iodimide, ioglicate, iothalamate, ioxithalamate, selectan, uroselectan, diodone, metrizoate, metrizamide, iohexyl, ioxaglate, iodixanol, lipidial, ethiodol and combinations thereof. Examples of MRI agents include, but are not limited to, gadolinium salts such as gadodiamide, gadopentetate, gadoteridol and gadoversetamide, superparamagnetic iron oxide particles, iron oxide compounds, and compounds of iron and manganese (in a 3+ oxidation state).

### EXAMPLE 1

For example, 100 mg of everolimus can be dissolved in 15 mL of chloroform to prepare a concentrated treatment agent solution. 50 mg of porous silica particles are added to prepare a 2:1 treatment agent:particle solution. After 30 minutes of moderate shaking, the solvent is evaporated by rotary evaporation for 60 minutes to yield treatment agent loaded porous particles. Finally, the particles are dried for 48 hours at 50° C. in an oven with a flow of nitrogen to remove trace amounts of solvent.

### EXAMPLE 2

In another example, 250 milliliters (mL) of solution is prepared containing 375 millimoles (mmol) of calcium nitrate tetrahydrate and 42 mmol of ferric nitrate nonahydrate. Another 250 mL of solution is prepared containing 250 mmol of diammonium hydrogen phosphate. The pH of the phosphate solution is adjusted to 8, and with stirring, the calcium/iron solution is added to the phosphate solution. After stirring overnight, the solids are isolated by centrifugation and rinsed with three, 250 ml portions of deionized water. After sintering at 70° C. for one hour, the calcium iron phosphate is ground to micron size particles in a ball mill. This results in biodegradable calcium phosphate microparticles, 1-10 μm in diameter, loaded with ferric (Fe3+) ions which can serve as a MRI contrast agent.

In some embodiments, the particle may be a porous microparticle with the capacity to carry preloaded particles which have been treated with a treatment agent and optionally an image-enhancing agent. For example, the porous microparticle can be in a range of about 150 nm to about 10 μm, preferably about 1 μm to about 3 μm, while the preloaded particle can be in the range of about 50 nm to about 8 μm, preferably about 50 nm to about 1 μm. The preloaded particle may or may not be porous.

In one example, the preloaded particle may be a porous nanoparticle 140 (including pores 140A and 140B) of glass, metal or ceramic representatively shown in FIG. 2B. In some embodiments, the pores of the nanoparticle can be loaded with at least one treatment agent. Thereafter, the loaded nanoparticle can be loaded into a microparticle. In some embodiments, the microparticle may thereafter be loaded with at least one treatment agent. In addition, the nanoparticle may optionally be loaded with an image-enhancing agent. Alternatively, the microparticle loaded with the treatment agent may be loaded directly with an image-enhancing agent.

In some embodiments a non-porous particle 150 (including pores 150A and 150B) of biocompatible glass, metal or ceramic may be a carrier, representatively shown in FIG. 2C. For example, a non-porous nanorod or nanofiber may be treated with a treatment agent. The treated non-porous particles may be used as the carrier, or, alternatively, may be loaded into a microparticle. In an embodiment in which porous or non-porous rod-shaped particles are used as the carrier, it is believed that the particles will have increased retention on the walls of the treatment site 110. Due to their shape, which may be similar to that of blood platelets, i.e., 2 and 4 μm, the rod-shaped particles will have a tendency to be pushed toward the walls of the blood vessel by larger-sized blood components and the turbulent flow in the center of the blood vessel. Contributing factors to this phenomenon also include hematocrit, shear rate, erythrocyte deformation and tube diameter. “Hematocrit” is the percentage of red blood cells in the blood. “Shear rate” is the rate at which adjacent layers of fluid move with respect to one another and is usually expressed in reciprocal seconds. “Erythrocyte deformation” is the deformation of red blood cells caused by blood flow. The overall result of localized dispersion of all blood cell components is a near-wall excess of platelet-sized particles. As a result, the concentration of rod-shaped particles adjacent to the side of the blood vessel wall may be increased which may result in increased retention of the rod-shaped particles at the treatment site.

The particles, e.g., particles 130, 140 and 150, may be coated with a coating agent that can enhance uptake during delivery and contribute to sustained release of the particles. The coating agent, or sustained-release coating, may be a polymer with a water uptake factor of about 0.2% to about 2% (e.g., hydrophobic) or about 5% to about 100% (e.g., hydrophilic). Suitable materials for sustained-release coatings include, but are not limited to, encapsulation polymers such as poly(L-lactide), poly(D,L-lactide), poly (glycolide), poly(D,L-lactide-co-glycolide), Poly(L-lactide-co-glycolide), polycaprolactone, polyanhydride, polydioxanone, polyorthoester, poly(ester amide) (PEA), polyamino acids, or poly(trimethylene carbonate), and combinations thereof. In addition, sustained-release coatings include those components that breakdown through degradation or erosion at a different time after implantation. One example of a sustained release carrier composition response is a poly(D,L-lactide-co-glycolide) (PLGA) system. With this system, the rate of breakdown of the coating can be controlled through a selection of the copolymer ratio, the molecular weight of the polymer, thermal and post-processing history (or intrusion, etc.) and the presence of acid end group. A 50:50 copolymer ratio is usually considered to be rapidly degrading, while increased copolymer ratios in either direction result in reduced degradation rates because of a balance between increased hydrophobicity with higher poly(lactic acid) (PLA) content and increased crystallinity with higher poly(glycolic acid) (PGA) content. The rate of degradation can be increased with acid end groups and by reducing the molecular weight of the polymer.

In some embodiments, the particles, e.g., particles 130, 140 and 150, can be formulated into a coating which can be coated on, for example, an implantable medical device. Representative examples of implantable medical devices include, but are not limited to, self-expandable stents, balloon-expandable stents, micro-depot or micro-channel stents and grafts. Suitable coating matrices include, but are not limited to, calcium phosphates such as hydroxyapatite, dahlite, brushite, octacalcium phosphate, calcium sulphate, or tricalcium phosphate (TCP). In addition, ceramic alloys such as alumina, silica, zirconia, titania, or Bioglass® (available from NovaBone, Alchua, Fla.), or carbides, such as tungsten carbide, may also be suitable. In one embodiment, the particles can be loaded with a hydrophilic treatment agent, such as a low molecular weight hydrophilic drug or a hydrophilic protein or peptide, and coated on a stent for controlled release thereof. In some embodiments, iron or magnesium may be incorporated within the particles to increase bioabsorbability. In some embodiments, the particles can be mixed with polymers such as D,L-PLA to increase bioabsorbability.

In addition to those factors described previously, other factors which can influence sustained release of a treatment agent in coatings consisting of bioabsorbable particles include, but are not limited to, volume fraction of coating within the pores, coating thickness and the use of rate-limiting topcoats (outer coating). For example, in one embodiment, a topcoat of about 25-200 μg/cm² of D,L-PLA, PEA, PVDF-HFP or PBMA may be used.

Methods of Manufacture

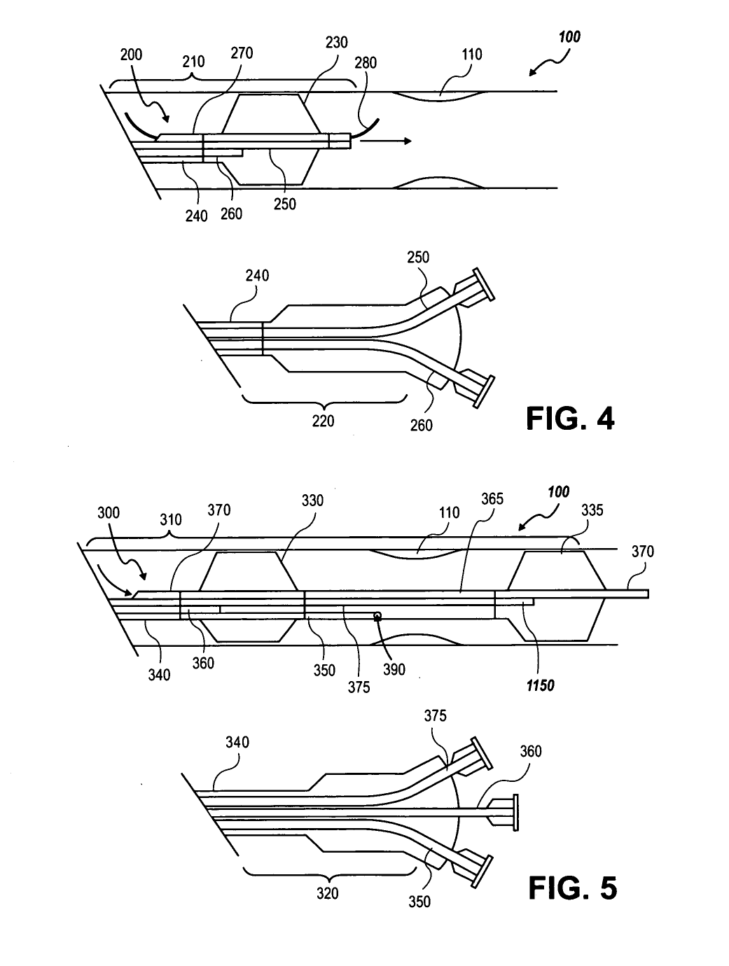

The bioabsorbable carriers of the present invention can be prepared for delivery to a treatment site by a variety of methods. FIG. 3 is a block diagram representing one method for preparing the carriers of FIGS. 2A-2C for sustained-release of at least one treatment agent. In this embodiment, the porous glass or metal particles are loaded with at least one treatment agent and optionally an image-enhancing agent (block 160). The more porous and tortuous the particle, the higher the loading capacity as explained previously in paragraph [0027]. To increase absorptivity at a treatment site, the particles can be coated with a sustained-release coating (block 170). The coated particles can then be suspended in a delivery solution (block 180), such as phosphate-buffered saline (PBS). In some embodiments, the treatment agent may be absorbed onto rather than loaded on the particle, especially in the embodiment in which non-porous bioabsorbable carriers are use. The resultant suspension may then be delivered to a treatment site. In some embodiments, a treatment agent is loaded into the porous carrier particle or coating with one or more excipients such as surfactants, phospholipids, sphingolipids, polymers, salts or any combination thereof.

In implantable medical device embodiments, a variety of methods may be used to coat stents or grafts with ceramic or glassy coatings, i.e., coatings which include bioabsorbable glass, metal and ceramic particles. In some embodiments, the treatment agent can be loaded within the pores of the particles when the treatment agent is in the porogen phase. “Porogen” is an agent that is incorporated in a substance to make the substance porous. In one method, a porogen is added to a substance and the substance is fabricated into a desired part. The porogen can be subsequently leached out resulting in a porous substance. In some embodiments, the porogen may be removed by using a selective solvent that only dissolves the porogen and not the substance. Suitable solvents include water and alcohol-based solvents. In some embodiments, heat is used to stabilize the materials by pyrolysis. This requires an agent which is thermally stable. Example of such agents would be MRI contrast agents, such as superparamagnetic iron oxide particles. Thus, when in the porogen phase, the treatment agent can be loaded within the pores of ceramic particles during synthesis, deposition, precipitation or sintering of the ceramic. For example, if the porogen is an organic material, such as dextrose, and the agent is temperature stable, such as iron oxide, sintering the ceramic can remove the porogen while leaving the iron oxide in the ceramic. Moreover, when the treatment agent is in the porogen phase, and the porogen is to be removed by solvent leaching, the goal is to leach out the porogen, while leaving the active agent behind. This may be accomplished by using several processes. For example, a solvent may be chosen where the porogen is soluble in the solvent, but the agent is not. Other porogens include, but are limited to salts, such as sodium and magnesium salts.

In some embodiments, a treatment agent saturated solution can be forced into the pores of particles by high pressure or by vacuum. For example, the solution can be added to, for example, ceramic particles in a Buchner funnel attached to a vacuum assembly. When the vacuum assembly is activated, the solution will be forced into the pores of the particles resulting in treatment agent loaded ceramic particles. In some embodiments, a stent may be coated with bioabsorbable particles without treatment agent. The stent may then be conditioned under vacuum infiltration with a treatment agent saturated solution.

In some embodiments, the particles and/or coating may be exposed to a molten solution of a neat treatment agent, i.e., drug. “Molten” means at a temperature high enough for the agent to be in a state fluid enough to allow flow. Conducting the process in an inert gas environment of nitrogen, argon, or vacuum, with no water or oxygen present, can enhance the stability of the treatment agent to the process. A “neat drug” is an undiluted drug without any additives. A vacuum may then be applied to the particles and/or coating (on a stent) with an inert gas, such as nitrogen. In this manner, the treatment agent can infiltrate the pores of the particles.

In some embodiments, the particles can be modified to include a substance with a chemical property that allows for a treatment agent to be loaded on or within the porous particle through a chemical interaction. For example, a particle can be modified such that it has an ionic property on the surface or within the pores. Thus, in embodiments in which the treatment agent is ionic, the treatment agent can be loaded by an ion-exchange process. An example of this would be a porous particle of hydroxyapatite. Anionic compounds such as oligonucleotides or DNA can be ion exchanged onto the particle surfaces. Other chemical properties which allow for treatment agent loading through chemical interactions, include, but are not limited to, hydrogen bonding, Van-der-Waals interaction, affinity interactions or combinations thereof.

In some embodiments, bioabsorbable glass, metal and ceramic particles are embedded within a polymer matrix. The polymer matrix may be used as a coating on an implantable device or as the matrix comprising at least some portion of an implantable device. In one embodiment, the polymer matrix is biodegradable or bioerodible.

In one embodiment, an implantable medical device, such as a stent, may be coated with a primer layer (inner coating) such as poly(butylmethacrylate). Bioabsorbable porous particles may then be applied to the stent by a process such as spraying. The spraying solution may contain the treatment agent. The stent may be dipped into an aqueous solution of treatment agent, infiltrating the pores and/or surfaces of the particles with treatment agent. An optional hydrophilic layer (outer coating) may then be applied. Examples of stent coating methods may be found in U.S. Pat. Nos. 6,506,437 to Harish et al., 6,733,768 to Hossainy et al., 6,712,845 to Hossainy et al., 6,713,119 to Hossainy et al., 6,759,054 to Chen et al. and 6,790,228 to Hossainy et al., hereby incorporated by reference.

Delivery Systems

A variety of delivery systems can be used to deliver the at least one treatment agent to a treatment site using bioabsorbable particles. The delivery systems include regional, local and direct injection systems in addition to stent deployment systems.

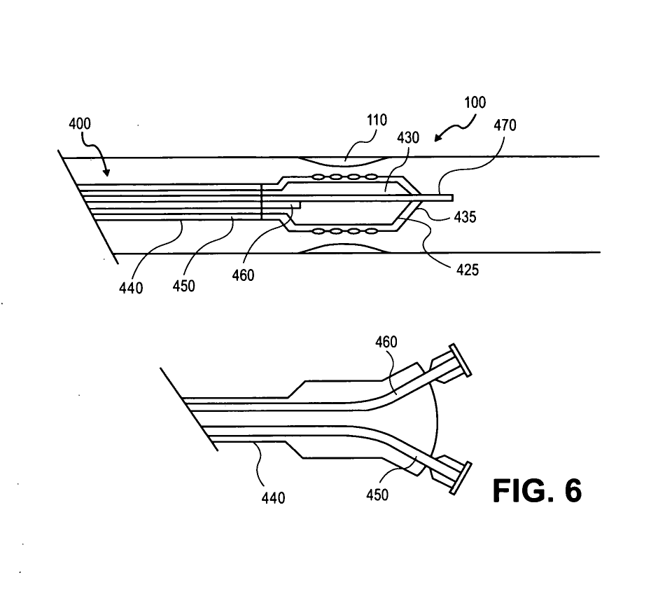

FIG. 4 shows blood vessel 100 having catheter assembly 200 disposed therein. Catheter assembly 200 includes proximal portion 220 and distal portion 210. Proximal portion 220 may be external to blood vessel 100 and to the patient during delivery of a treatment agent. Representatively, catheter assembly 200 may be inserted through a femoral artery and through, for example, a guide catheter and with the aid of a guidewire to a location in the vasculature of a patient. That location may be, for example, a coronary artery. FIG. 4 shows distal portion 210 of catheter assembly 200 positioned proximal or upstream from treatment site 110.

In one embodiment, catheter assembly 200 includes primary cannula 240 having a length that extends from proximal portion 220 (e.g., located external through a patient during a procedure) to connect with a proximal end or skirt of balloon 230. Primary cannula 240 has a lumen therethrough that includes inflation cannula 260 and delivery cannula 250. Each of inflation cannula 260 and delivery cannula 250 extends from proximal portion 220 of catheter assembly 200 to distal portion 210. Inflation cannula 260 has a distal end that terminates within balloon 230. Delivery cannula 250 extends through balloon 230.

Catheter assembly 200 also includes guidewire cannula 270 extending, in this embodiment, through balloon 230 through a distal end of catheter assembly 200. Guidewire cannula 270 has a lumen sized to accommodate guidewire 280. Catheter assembly 200 may be an over the wire (OTW) configuration where guidewire cannula 270 extends from a proximal end (external to a patient during a procedure) to a distal end of catheter assembly 200. Guidewire cannula 230 may also be used for delivery of a substance such as a bioabsorbable metal, glass or ceramic particle loaded with at least one treatment agent and optionally an image-enhancing agent when guidewire 280 is removed with catheter assembly 200 in place. In such case, separate delivery cannula (delivery cannula 250) is unnecessary or a delivery cannula may be used to deliver one substance while guidewire cannula 270 is used to delivery another substance.

In another embodiment, catheter assembly 200 is a rapid exchange (RX) type catheter assembly and only a portion of catheter assembly 200 (a distal portion including balloon 230) is advanced over guidewire 280. In an RX type of catheter assembly, typically, the guidewire cannula/lumen extends from the distal end of the catheter to a proximal guidewire port spaced distally from the proximal end of the catheter assembly. The proximal guidewire port is typically spaced a substantial distance from the proximal end of the catheter assembly. FIG. 4 shows an RX type catheter assembly.

In one embodiment, catheter assembly 200 is introduced into blood vessel 100 and balloon 230 is inflated (e.g., with a suitable liquid through inflation cannula 260) to occlude the blood vessel. Following occlusion, a solution (fluid) including a bioabsorbable glass, metal or ceramic particles loaded with at least one treatment agent and optionally an image-enhancing agent is introduced through delivery cannula 250. A suitable solution of treatment agent is a saline solution with a concentration of particles in the range of about 0.5% to about 5%, preferably about 1% to about 2%. By introducing the solution, the particles with treatment agent can absorb on the walls of the blood vessel at treatment site 110. It should be understood that the concentration will be at least partially dependent on the size of the particles and the viscosity of the solution.

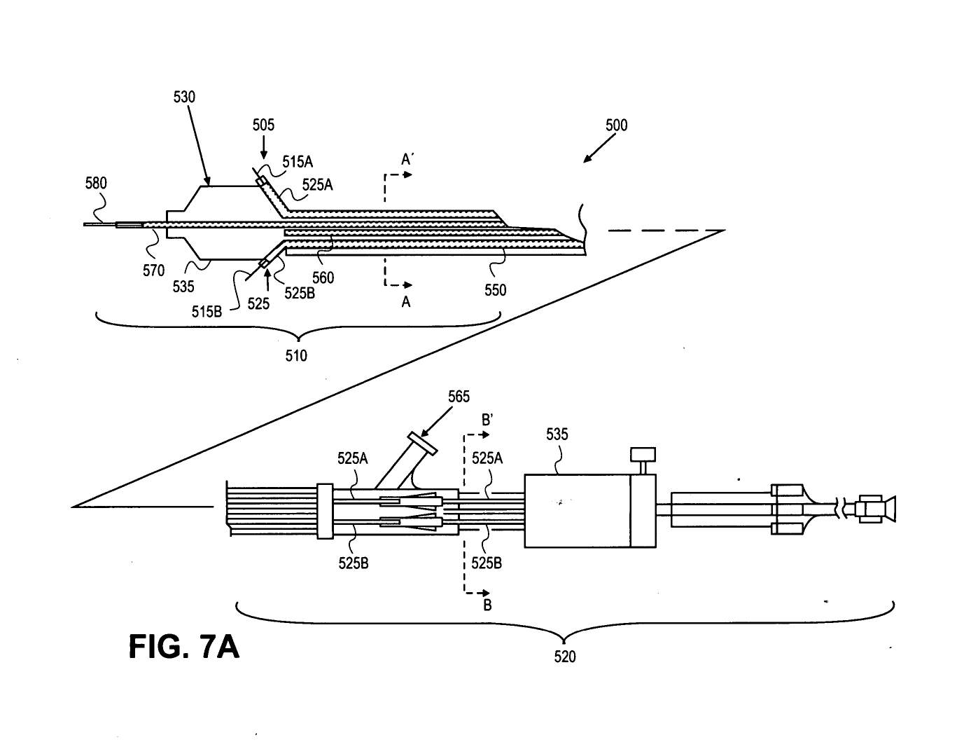

In an effort to improve the target area of bioabsorbable particles to a treatment site, such as treatment site 110, the injury site may be isolated prior to delivery. FIG. 5 shows an embodiment of a catheter assembly having two balloons where one balloon is located proximal to treatment site 110 and a second balloon is located distal to treatment site 110. FIG. 5 shows catheter assembly 300 disposed within blood vessel 100. Catheter assembly 300 has a tandem balloon configuration including proximal balloon 330 and distal balloon 335 aligned in series at a distal portion of the catheter assembly. Catheter assembly 300 also includes primary cannula 340 having a length that extends from a proximal end of catheter assembly 300 (e.g., located external to a patient during a procedure) to connect with a proximal end or skirt of balloon 330. Primary cannula 340 has a lumen therethrough that includes first inflation cannula 360 and second inflation cannula 375. First inflation cannula 360 extends from a proximal end of catheter assembly 300 to a point within balloon 330. First inflation cannula 360 and second inflation cannula 375 have lumens therethrough allowing balloon 330 and balloon 335 to be inflated, respectively. Thus, in this embodiment, balloon 330 is inflated through an inflation lumen separate from the inflation lumen that inflates balloon 335. First inflation cannula 360 has a lumen therethrough allowing fluid to be introduced in the balloon 330 to inflate the balloon. In this manner, balloon 330 and balloon 335 may be separately inflated. Each of first inflation cannula 360 and second inflation cannula 375 extends from, in one embodiment, the proximal end of catheter assembly 300 through a point within balloon 330 and balloon 335, respectively.

Catheter assembly 300 also includes guidewire cannula 370 extending, in this embodiment, through each of balloon 330 and balloon 335 through a distal end of catheter assembly. Guidewire cannula 370 has a lumen therethrough sized to accommodate a guidewire. No guidewire is shown within guidewire cannula 370. Catheter assembly 300 may be an over the wire (OTW) configuration or a rapid exchange (RX) type catheter assembly. FIG. 5 illustrates an RX type catheter assembly.

Catheter assembly 300 also includes delivery cannula 350. In this embodiment, delivery cannula 350 extends from a proximal end of catheter assembly 300 through a location between balloon 330 and balloon 335. Secondary cannula 365 extends between balloon 330 and balloon 335. A proximal portion or skirt of balloon 335 connects to a distal end of secondary cannula 365. A distal end or skirt of balloon 330 is connected to a proximal end of secondary cannula 365. Delivery cannula 350 terminates at opening 390 through secondary cannula 365. In this manner, bioabsorbable metal, glass or ceramic particles may be introduced between balloon 330 and balloon 335 positioned adjacent to treatment site 110.

FIG. 5 shows balloon 330 and balloon 335 each inflated to occlude a lumen of blood vessel 100 and isolate treatment site 110. In one embodiment, each of balloon 330 and balloon 335 are inflated to a point sufficient to occlude blood vessel 100 prior to the introduction of bioabsorbable metal, glass or ceramic particles. The particles loaded with at least one treatment agent and optionally an image-enhancing agent may then be introduced.

In the above embodiment, separate balloons having separate inflation lumens are described. It is appreciated, however, that a single inflation lumen may be used to inflate each of balloon 330 and balloon 335. Alternatively, in another embodiment, balloon 330 may be a guidewire balloon configuration such as a PERCUSURG™ catheter assembly where catheter assembly 300 including only balloon 330 is inserted over a guidewire including balloon 335.

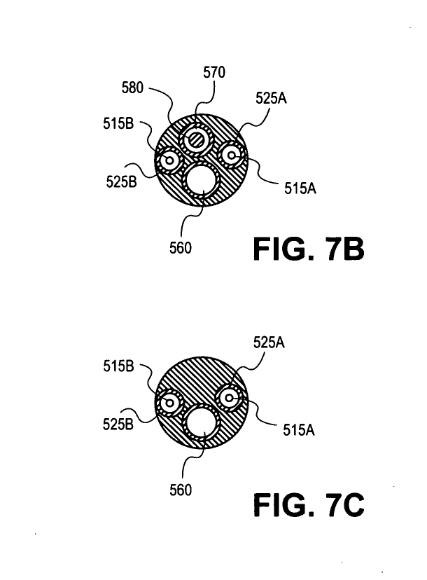

FIG. 6 shows another embodiment of a catheter assembly. Catheter assembly 400, in this embodiment, includes a porous balloon through which a substance, such as bioabsorbable metal, glass or ceramic particles loaded with at least one treatment agent and optionally an image-enhancing agent, may be introduced. FIG. 6 shows catheter assembly 400 disposed within blood vessel 100. Catheter assembly 400 has a porous balloon configuration positioned at treatment site 110. Catheter assembly 400 includes primary cannula 440 having a length that extends from a proximal end of catheter assembly 400 (e.g., located external to a patient during a procedure) to connect with a proximal end or skirt of balloon 430. Primary cannula 440 has a lumen therethrough that includes inflation cannula 460. Inflation cannula 460 extends from a proximal end of catheter assembly 400 to a point within balloon 430. Inflation cannula 460 has a lumen therethrough allowing balloon 430 to be inflated through inflation cannula 460.

Catheter assembly 400 also includes guidewire cannula 470 extending, in this embodiment, through balloon 430. Guidewire cannula 470 has a lumen therethrough sized to accommodate a guidewire. No guidewire is shown within guidewire cannula 470. Catheter assembly 400 may be an over-the-wire (OTW) configuration or rapid exchange (RX) type catheter assembly. FIG. 6 illustrates an OTW type catheter assembly.

Catheter assembly 400 also includes delivery cannula 450. In this embodiment, delivery cannula 450 extends from a proximal end of catheter assembly 400 to proximal end or skirt of balloon 430. Balloon 430 is a double layer balloon. Balloon 430 includes inner layer 425 that is a non-porous material, such as PEBAX, Nylon or PET. Balloon 430 also includes outer layer 435. Outer layer 435 is a porous material, such as expanded poly(tetrafluoroethylene) (ePTFE). In one embodiment, delivery cannula 450 is connected to between inner layer 425 and outer layer 435 so that a substance can be introduced between the layers and permeate through pores in balloon 430 into a lumen of blood vessel 100.

As illustrated in FIG. 6, in one embodiment, catheter assembly is inserted into blood vessel 100 so that balloon 430 is aligned with treatment site 110. Following alignment of balloon 430 of catheter assembly 400, balloon 430 may be inflated by introducing an inflation medium (e.g., liquid through inflation cannula 460). In one embodiment, balloon 430 is only partially inflated or has an inflated diameter less than an inner diameter of blood vessel 100 at treatment site 110. In this manner, balloon 430 does not contact or only minimally contacts the blood vessel wall. A suitable expanded diameter of balloon 430 is on the order of 2.0 to 5.0 mm for coronary vessels. It is appreciated that the expanded diameter may be different for peripheral vasculature. Following the expansion of balloon 430, a substance, such as bioabsorbable glass, metal or ceramic particles loaded with at least one treatment agent and optionally an image-enhancing agent is introduced into delivery cannula 450. The treatment agent flows through delivery cannula 450 into a volume between inner layer 425 and outer layer 435 of balloon 430. At a relatively low pressure (e.g., on the order of two to four atmospheres (atm)), the bioabsorbable particles then permeate through the porous of outer layer 430 into blood vessel 100.

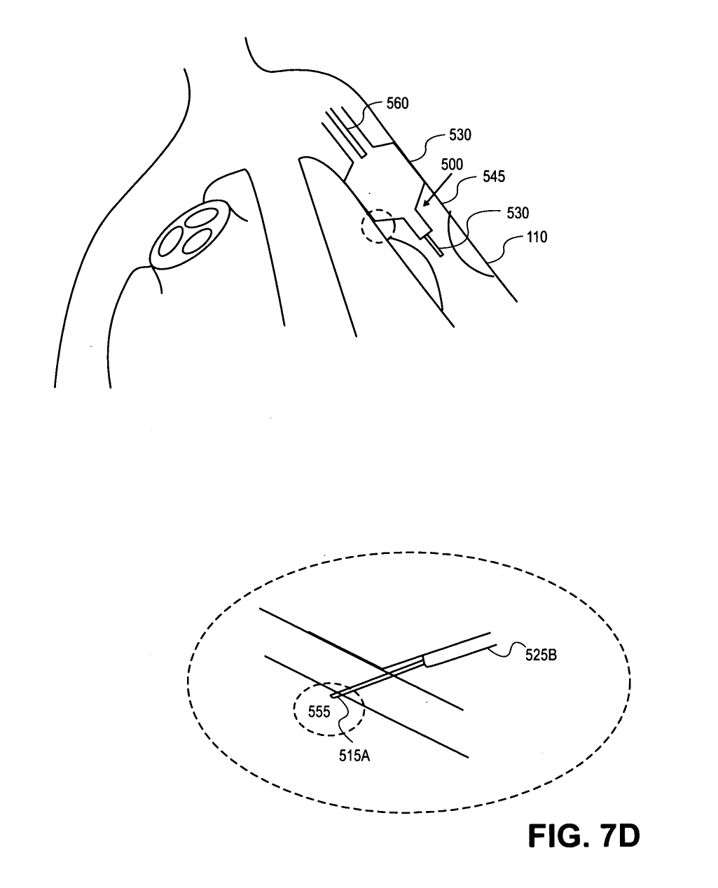

FIGS. 7A-D illustrate an alternative embodiment of a catheter assembly. In general, the catheter assembly 500 provides a system for delivering a substance, such as bioabsorbable glass, metal or ceramic particles loaded with at least one treatment agent and optionally an image-enhancing agent, to or through a desired area of a blood vessel (a physiological lumen) or tissue in order to treat a localized area of the blood vessel or to treat a localized area of tissue possibly located adjacent to the blood vessel. The catheter assembly 500 is similar to the catheter assembly 500 described in commonly-owned, U.S. Pat. No. 6,554,801, titled “Directional Needle Injection Drug Delivery Device”, and incorporated herein by reference.

In one embodiment, catheter assembly 500 is defined by elongated catheter body 550 having proximal portion 520 and distal portion 510. FIG. 7B shows catheter assembly 500 through line A-A′ of FIG. 7A (at distal portion 510). FIG. 7C shows catheter assembly 500 through line B-B′ of FIG. 7A.

Guidewire cannula 570 is formed within catheter body (from proximal portion 510 to distal portion 520) for allowing catheter assembly 500 to be fed and maneuvered over guidewire 580. Balloon 530 is incorporated at distal portion 510 of catheter assembly 500 and is in fluid communication with inflation cannula 560 of catheter assembly 500.

Balloon 530 can be formed from balloon wall or membrane 335 which is selectively inflatable to dilate from a collapsed configuration to a desired and controlled expanded configuration. Balloon 530 can be selectively dilated (inflated) by supplying a fluid into inflation cannula 560 at a predetermined rate of pressure through inflation port 565. Balloon wall 335 is selectively deflatable, after inflation, to return to the collapsed configuration or a deflated profile. Balloon 530 may be dilated (inflated) by the introduction of a liquid into inflation cannula 560. Liquids containing treatment and/or diagnostic agents may also be used to inflate balloon 530. In one embodiment, balloon 530 may be made of a material that is permeable to such treatment and/or diagnostic liquids (see FIG. 6). To inflate balloon 530, the fluid can be supplied into inflation cannula 560 at a predetermined pressure, for example, between about one and 20 atmospheres. The specific pressure depends on various factors, such as the thickness of balloon wall 335, the material from which balloon wall 335 is made, the type of substance employed and the flow-rate that is desired.

Catheter assembly 500 also includes substance delivery assembly 505 for injecting a substance into a tissue of a physiological passageway. In one embodiment, substance delivery assembly 505 includes needle 515a movably disposed within hollow delivery lumen 525a. Delivery assembly 505 includes needle 515b movably disposed within hollow delivery lumen 525b. Delivery lumen 525a and delivery lumen 525b each extend between distal portion 510 and proximal portion 520. Delivery lumen 525a and delivery lumen 525b can be made from any suitable material, such as polymers and copolymers of polyamides, polyolefins, polyurethanes, and the like. Access to the proximal end of delivery lumen 525a or delivery lumen 525b for insertion of needle 515a or 515b, respectively is provided through hub 535.

One or both of delivery lumen 525a and delivery lumen 525b may be used to deliver a substance to a treatment site. Alternatively, one delivery lumen (e.g., delivery lumen 525a) may be used to deliver one substance while the other delivery lumen (e.g., delivery lumen 525b) may be used to deliver another substance.

FIG. 7D illustrates one technique using catheter assembly to deliver bioabsorbable glass, metal or ceramic particles loaded with at least one treatment agent and optionally an image-enhancing agent to tissue and/or an organ. In a typical procedure, guidewire 580 is introduced into, for example, arterial system of the patient (e.g., through the femoral artery) until the distal end of guidewire 580 is upstream of the narrowed lumen of the blood vessel (e.g., upstream of occlusion 110). Catheter assembly 500 is mounted on the proximal end of guidewire 580 and translated down guidewire 580 until catheter assembly 500 is positioned as desired. In the example shown in FIG. 7D, catheter assembly 500 is positioned so that balloon 530 and delivery cannula 550 are upstream of the narrowed lumen of left circumflex artery (LCX) 545. Angiographic techniques may be used to place catheter assembly 500.

In the embodiment shown in FIG. 7D, needle 515a is advanced through the wall of LCX 545 to peri-adventitial site 555. Needle 515a is placed at a safe distance, determined by the measurement of a thickness of the blood vessel wall and the proximity of the exit of delivery cannula 525a to the blood vessel wall. Adjustment knob 565 (see FIG. 7A) may be used to accurately locate needle tip 515a in the desired peri-adventitial region. Once in position, a substance, such as bioabsorbable glass, metal or ceramic particles loaded with at least one treatment agent and optionally an image-enhancing agent is introduced through needle 515a to the treatment site (peri-adventitial site 555).

In the above described embodiment of locating a treatment agent within or beyond a blood vessel wall (e.g., at a peri-adventitial site), it is appreciated that an opening is made in or through the blood vessel. In same instances, it may be desirable to plug or fill the opening following delivery of the treatment agent. This may be accomplished by introduction through a catheter lumen of cyanoacrylate or similar material that will harden on contact with blood.

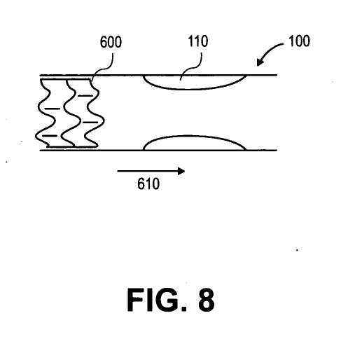

FIG. 8 illustrates an alternative delivery system for delivery of bioabsorbable glass, metal or ceramic particles loaded with at least one treatment agent and optionally an image-enhancing agent using a stent. In one embodiment, a stent 600 can be deployed in a blood vessel 100 upstream from the treatment site 110. Stent deployment methods are known by those skilled in the art. The stent 600 can be coated with bioabsorbable glass, metal or ceramic particles loaded with at least one treatment agent. In some embodiments, the particles can be loaded into depots of a depot stent. In some embodiments, the stent can be coated with a sustained-release coating similar to that employed in coating the particles and more fully described in paragraph [0036]. Over time, the stent 600 can “shed” the particles which can adhere to the treatment site 110 as they wash downstream (arrow 610). The shedding can be attributed to a variety of factors, including the degradation and/erosion of the sustained-release coating and the natural flow of blood exerting force on the particles over time.

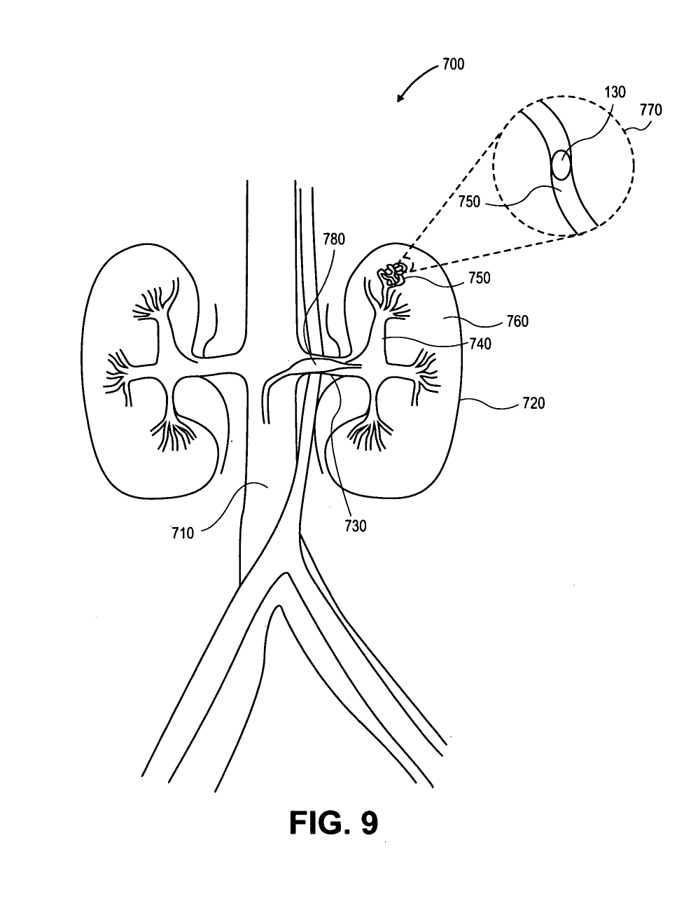

FIG. 9 is a schematic illustration 700 of a back view of the kidneys and renal blood vessels of the body. A lower branch of aorta 710 feeds blood to kidneys 720 through renal artery 730. Renal artery 730 branches off into arteriole 740 which in turn lead to a capillary tuft 750, hereinafter interchangeably referred to as a glomerulus. Blood from arteriole 760 flows into glomerulus 750 where it is filtered to remove fluid and solutes from the blood.

A delivery system, such as those described in relation to FIGS. 4-7, can be positioned within renal artery 730. Bioabsorbable glass, metal or ceramic particles loaded with at least one treatment agent and optionally an image-enhancing agent within a catheter may be released into renal artery 730 such that they flow through arteriole 740 and into glomerulus 750. In one embodiment, the particles may have a diameter such that they become lodged within a narrow lumen of glomerulus 750. In this aspect, as the particles degrade over time, treatment agent is released and localized to glomerulus 750.

From the foregoing detailed description, it will be evident that there are a number of changes, adaptations and modifications of the present invention which come within the province of those skilled in the part. The scope of the invention includes any combination of the elements from the different species and embodiments disclosed herein, as well as subassemblies, assemblies and methods thereof. However, it is intended that all such variations not departing from the spirit of the invention be considered as within the scope thereof.

1. A method comprising:
percutaneously introducing a delivery device into a blood vessel from a point outside a patient; and
delivering at least one substance to a treatment site within a lumen of a blood vessel by a sustained-release carrier.

2. The method of claim 1, wherein the carrier is selected from the group consisting of a bioabsorbable glass, a bioabsorbable metal and a bioabsorbable ceramic.

3. The method of claim 2, wherein the carrier is porous.

4. The method of claim 2, wherein, the carrier is nonporous.

5. The method of claim 2, wherein the carrier is from 10 nanometers to 10 microns in size.

6. The method of claim 2, wherein the carrier is selected from the group consisting of rod-shaped and platelet-shaped carriers.

7. The method of claim 1, wherein the substance is at least one selected from the group consisting of a biological component, a treatment agent and an image-enhancing agent.

8. The method of claim 7, wherein the biological component is one selected from the group consisting of an antibody, an affibody, an aptamer, a protein, a peptide and an oligonucleotide.

9. The method of claim 7, wherein the treatment agent is one selected from the group consisting of an anti-inflammatory, an anti-platelet, an anti-coagulant, an anti-fibrin, an anti-thrombonic, an anti-mitotic, an anti-biotic, an anti-allergic, an anti-oxidant, an anti-proliferative and an anti-migratory.

10. The method of claim 7, wherein the image-enhancing agent is one selected from the group consisting of a radiopaque dye and a magnetic resonance imaging agent.

11. The method of claim of 1, wherein the carrier is coated with a sustained-release coating substance.

12. The method of claim 11, wherein the coating substance is one selected from the group consisting of a poly(L-lactide), a poly(D,L-lactide), a poly(glycolide), a poly(D,L-lactide-co-glycolide), poly(L-lactide-co-glycolide), a polycaprolactone, a polyanhydride, a polydioxanone, a polyorthoester, a polyamino acid, a poly(ester amide), a poly(trimethylene carbonate) and combinations thereof.

13. The method of claim 3, wherein the substance is present in at least one pore of the carrier.

14. The method of claim 13, wherein the carrier comprises a first carrier that, at the time of delivery, is present within the pore of a second carrier.

15. The method of claim 14, wherein the second carrier comprises a microparticle.

16. A method of manufacturing a composition comprising:
loading a substance into a carrier;
coating the carrier with a coating substance; and
suspending the carrier in a solution.

17. The method of claim 16, wherein the carrier is one selected from the group consisting of porous bioabsorbable glass, bioabsorbable metal and bioabsorbable ceramic.

18. The method of claim 16, wherein the substance is at least one selected from the group consisting of a biological component, a treatment agent and an image-enhancing agent.

19. The method of claim 16, wherein the coating substance is one selected from the group consisting of a poly(L-lactide), a poly(D,L-lactide), a poly(glycolide), a poly(D,L-lactide-co-glycolide), a poly(L-lactide-co-glycolide), a polycaprolactone, a polyanhydride, a polydioxanone, a polyorthoester, a poly(ester amide), a polyamino acid, a poly(trimethylene carbonate) and combinations thereof.

20. The method of claim 19, wherein the coating has a water uptake from 0.2 percent to 100 percent.

21. A composition comprising:
one of a bioabsorbable metal, glass and ceramic carrier; and
a substance loaded within the bioabsorbable carrier.

22. The composition of claim 21, wherein the bioabsorbable metal, glass, ceramic carrier comprises a shape of one of a bead, a rod or a fiber.

23. The composition of claim 21, wherein the substance is at least one selected from the group consisting of a biological component, a treatment agent and an image-enhancing agent.

24. The composition of claim of 21, wherein the carrier is coated with a sustained-release coating substance.

25. The method of claim 24, wherein the coating substance is one selected from the group consisting of a poly(L-lactide), a poly(D,L-lactide), a poly(glycolide), a poly(D,L-lactide-co-glycolide), a poly(L-lactide-co-glycolide), a polycaprolactone, a polyanhydride, a polydioxanone, a polyorthoester, a poly(ester amide), a polyamino acid, a poly(trimethylene carbonate) and combinations thereof.

26. An implantable medical device comprising:
a stent;
a treatment agent; and
a sustained-release coating comprising at least one porous carrier selected from the group consisting of a bioabsorbable glass, a bioabsorbable metal and a bioabsorbable ceramic, wherein the treatment agent is dispersed within at least one pore of the porous carrier.

27. The device of claim 26, wherein the bioabsorbable ceramic is selected from the group consisting of a calcium phosphate, a carbide and a ceramic alloy.

28. The device of claim 26, further comprising an inner coating and an outer coating, wherein the sustained-release coating is between the inner coating and the outer coating.

29. The device of claim 28, wherein the inner coating comprises polybutylmethacrylate.

30. The device of claim 28, wherein the outer coating comprises at least one of polylactide, poly(ester amide), polyethylene glycol and poly(vinyldiene fluoride-co-hexafluoropropylene).

31. The device of claim 30, wherein the outer coating has a sustained-release characteristic.

32. The device of claim 26, wherein the treatment agent is hydrophilic.

33. The method of claim 8, wherein the component is Apo A1 protein.
